(19) United States
(12) Patent Application Publication
(10) Pub. No.: US 2013/0034502 A1
(43) Pub. Date: Feb. 7, 2013

(54) Color Changing Cosmetic

(71) Applicants: Jian-an Andy LAI, Troy, NY (US); Richard Dominick TIMPERIO, Catskill, NY (US)

(72) Inventors: Jian-an Andy LAI, Troy, NY (US); Richard Dominick TIMPERIO, Catskill, NY (US)

(21) Appl. No.: 13/562,338

(22) Filed: Jul. 31, 2012

Related U.S. Application Data
(60) Provisional application No. 61/515,362, filed on Aug. 5, 2011.

(51) Int. Cl. A61K 49/00; A61Q 5/06; A61Q 1/02; A61K 8/00

(52) U.S. Cl. 424/9.8; 424/70.6; 424/63

(57) ABSTRACT

The present invention is a substance that allows users to monitor temperature changes and appearance cosmetically on the human body by a change in color of the applied substance. It is intended for direct application to the skin and hair. The range of colors can be adjusted depending on the demand for the specific product or desired effect. The product is meant to be a temporary solution to cosmetically visually show the users temperature or change how the user looks. The cosmetic substance can be applied in many different ways and its unique color changing characteristics can be used on many products.

### CROSS-REFERENCE TO RELATED APPLICATIONS

This application claims priority from U.S. Provisional Application Ser. No. 61/515,362, filed on Aug. 5, 2011, the entirety of which is hereby expressly incorporated by reference herein.

### FIELD OF THE INVENTION

The present invention relates to a color changing body cosmetic and more specifically to a topical skin and hair treatment which changes colors with the changes in body temperature.

### BACKGROUND OF THE INVENTION

In the use of skin and hair products, depending on the style and individual, there are different cosmetic variations that are utilized to change or enhance ones appearance. These cosmetics can include makeup, paint and other types of product designed to alter the look of a person's face, among others.

There are many reasons when people use these types of cosmetic products to alter their look. Cosmetics also have numerous purposes including self-expression, identification, ritual, and advertising. Some may be used for special events. For example, the temporary body paint which may be used at a sporting event like a football game or face paint which may be used at a child's party. Some use cosmetics for daily use, such as hair coloring. In these cases as well as others, it is appropriate for the cosmetic effects not to be permanent.

In prior art of cosmetic skin and hair products, there are many colors and pigments available. The color of the product is designed in such a way as to maintain the color attribute. As an example, a body paint that is the color ‘Red’ would be applied to the skin and would be the visible as the color ‘Red’.

All of these products fail to teach or suggest a skin and hair cosmetic that changes in color.

Therefore, it is a desirable to develop a skin and hair cosmetic that will automatically change in color and provide a unique style based in the individual.

### SUMMARY OF THE INVENTION

Thus, according to one aspect of the present invention, a cosmetic applied to the face, body or hair would be applied and would dynamically change colors based on the temperature of the face, body or hair respectively.

According to another aspect of the present invention, the color of the cosmetic is temperature sensitive and will change color as to the environmental ambient temperature it is exposed to. As related to one of its uses in regards to hair, there would be a color change effect as a range of colors. The cosmetic as applied to the hair would be effected by both the temperature of the individual where the hair is near the persons head, and the surrounding environment temperature where the hair is furthest away from the persons head. In these cases this creates a multi-color effect that changes for a unique situation based on both body temperature and surrounding temperature.

Additionally there are other characteristics to the color of the cosmetics were in some cases the base topical cosmetic does not have a color and is transparent in certain temperature ranges. As to state an example, the cosmetic will not be apparent by sight and is invisible at normal body temperature, however when the body temperature is elevated as when exercising or when outside in the direct sun, the cosmetic will then start to appear and change to visible colors. Yet another example of this would be the use on hair. When applied in a normal inside room temperature the cosmetic is clear, however once exposed to the outside sunlight the color would appear and be visible.

According to still another aspect of the present invention, the temperature sensitive color change would be applied to temporary tattoos. In this case a temporary tattoo would be applied to the body and the colors would change from the base color to the representative color for their skin temperature. This would present a unique range of colors for different individuals.

In an alternative embodiment, the cosmetic substance can be applied to an individual to monitor their body temperature in the case of sickness or injury. The resulting color change of the cosmetic would quickly and easily produce a visual indicator of the body temperature. In the event of injury to a specific area of the body, the substance could be applied to that area to help professionals diagnose by showing the temperature by the injury.

Numerous other aspects, features and advantages of the present invention will be made apparent from the following detailed description taken together with the drawing figures.

### BRIEF DESCRIPTION OF THE DRAWINGS

The drawings illustrate the best mode currently contemplated for practicing the present invention.

In the drawings:

FIG. 1 is a front view of a human person with references to example locations for use of the color changing cosmetic.

### DETAILED DESCRIPTION OF THE INVENTION

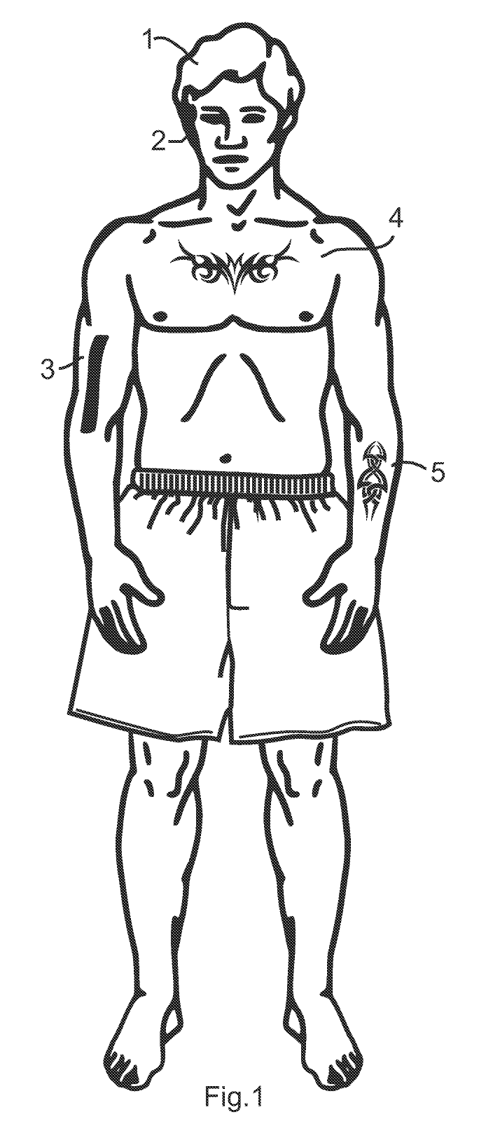

With reference to the drawing in which numerals designate areas throughout the disclosure, the present invention is indicated generally in FIG. 1.

The mentioned invention is a cosmetic substance used to enhance the appearance of the human which will alter or change its color based on the human and surrounding temperature. This substance also can change from a visible color to clear or clear to a visible color based on the human and surrounding temperature. The cosmetic substance includes but is not limited to, skin cream, lotions, gels, powders, hair sprays, body paints and gels. Generally the application areas would be the hair 1, face 2 and body 3.

Temporary tattoos 4 and 5 are a non-permanent image on the skin and can be applied by ink transfer, drawn, painted, stenciled or air brushed amount other methods. These mediums provide a method for application of the cosmetic substance in the mentioned invention. This would provide the color changing effect not currently shown by the existing substances. Temporary tattoos would be able to change to various colors based on body, hair and surrounding temperature.

The present invention will give an easy temporary solution for cosmetic use. The temperature sensitive cosmetic would change color when exposed to different temperature ranges. For example; (low temp=color ‘X’, mid temp=color ‘Y’, high temp=color ‘Z’). The substance would have various options/combinations to choose what color would appear at the various temperature ranges. E.g. when the consumer is waiting outside in the winter the hair color would be color ‘X’. After entering a building the room temperature would cause the color to change to ‘Y’. If in the gym, party or club and the body becomes active and rises above a certain temperature the color would change to color ‘Z.’ Depending on the application there is a color resolution in the temperature ranges.

The cosmetic substance is customizable to as far as the amount of color to temperature ranges and the colors that can be assigned to each temperature. This provides a truly individual effect and custom appearance.

What is claimed is:

1. A cosmetic substance which changes colors based on one or both of:
a. the temperature of the skin and body
b. the ambient temperature surrounding the individual

2. The cosmetic substance of claim 1 which can be used on the skin and hair.

3. The cosmetic substance of claim 1 which can have multiple methods of application.

4. The cosmetic substance of claim 1 which can be used to monitor the user by showing a color in response to their body temperature.

5. The cosmetic substance of claim 1 which in temporary and can be removed from the skin or hair without lasting effects.
